SEQUENCE LISTING (1 sequence)

SEQ ID NO: 1 - protein, 406 aa, Aspergillus aculeatus
MVGLLSITAALAATVLPNIVSAVGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

(19) United States
(12) Patent Application Publication
(10) Pub. No.: US 2012/0276585 A1
(43) Pub. Date: Nov. 1, 2012

(54) METHOD FOR PRODUCING FERMENTATION PRODUCTS FROM LIGNOCELLULOSE-CONTAINING MATERIAL

(75) Inventors: Haiyu Ren, Beijing (CN); Dongmin Li, Beijing (CN); Yun Wang, Beijing (CN); Chuanji Fang, Beijing (CN); Hong Zhi Huang, Beijing (CN)

(73) Assignees: COFCO CORPORATION, Beijing (CN); NOVOZYMES A/S, Bagsvaerd (DK)

(21) Appl. No.: 13/515,925

(22) PCT Filed: Dec. 21, 2010
(86) PCT No.: PCT/EP10/70338

§ 371 Date: Jul. 12, 2012

(30) Foreign Application Priority Data
Dec. 21, 2009 (CN) PCT/CN2009/075775

(51) Int. Cl. C12P 19/14; C12P 7/04; C12P 7/16; C12P 7/48; C12P 7/54; C12P 7/44; C12P 7/56; C12P 7/58; C12P 7/28; C12P 13/14; C12P 3/00; C12P 1/02; C12P 37/00; C12P 29/00; C12N 9/00; C12P 25/00; C12P 19/42; C12P 23/00; C12P 7/14

(52) U.S. Cl. 435/43; 435/99; 435/162; 435/157; 435/160; 435/144; 435/140; 435/142; 435/139; 435/137; 435/150; 435/110; 435/168; 435/171; 435/64; 435/183; 435/66; 435/86; 435/67

(57) ABSTRACT

The present invention relates to a method for producing a hydrolysate of from lignocellulose-containing material, comprising pre-treatment with low temperature, hydrolysis and fermentation, wherein hydrolysis is performed by contacting the lignocellulose-containing material with an enzyme composition comprising at least 10% xylanase enzyme protein w/w%.

### FIELD

Methods for producing fermentation products from lignocellulose-containing material, and more particularly, methods for enhancing the enzymatic hydrolysis of lignocellulose-containing material by a two-stage pre-treatment are disclosed.

### REFERENCE TO A SEQUENCE LISTING

This application contains a Sequence Listing in computer readable form. The computer readable form is incorporated herein by reference.

BACKGROUND

Lignocellulose-containing material, or biomass, may be used to produce fermentable sugars, which in turn may be used to produce fermentation products such as renewable fuels and chemicals. Lignocellulose-containing material is a complex structure of cellulose fibers wrapped in a lignin and hemicellulose sheath. Production of fermentation products from lignocellulose-containing material includes pre-treating, hydrolyzing, and fermenting the lignocellulose-containing material.

The structure of lignocellulose is not directly accessible to enzymatic hydrolysis. Therefore, the lignocellulose is pre-treated in order to break the lignin seal and disrupt the crystalline structure of cellulose. This may cause solubilization and saccharification of the hemicellulose fraction. The cellulose fraction is then hydrolyzed enzymatically, e.g., by cellulolytic enzymes, which degrades the carbohydrate polymers into fermentable sugars. These fermentable sugars are then converted into the desired fermentation product by a fermenting organism, which product may optionally be recovered, e.g., by distillation. Producing fermentation products from lignocellulose-containing material is currently very expensive. Consequently, there is a need for providing further processes for producing fermentation products from lignocellulose-containing materials.

### SUMMARY

The present invention relates to a method for producing a hydrolysate from a lignocellulose-containing material, comprising pre-treating the lignocellulose-containing material at a relatively low temperature, followed by hydrolysis with an enzyme composition comprising a high proportion of xylanase.

Accordingly, in an aspect the present invention relates to a process for producing a hydrolysate of a lignocellulosic material comprising (a) subjecting the lignocellulosic material to a pretreatment at a temperature between 165° C. and 175° C., (b) subjecting the pretreated lignocellulosic material to the action of hydrolytic enzymes to produce a hydrolysate, wherein the hydrolytic enzymes comprises cellulytic enzymes and a xylanase, said xylanase being present in an amount of at least 10% of the total amount hydrolytic enzyme protein.

One advantage of the present invention includes that the digestibility of lignocelluloses-containing material is improved significantly, therefore lowering the cost for producing fermentation products from lignocellulose-containing material.

### BRIEF DESCRIPTION OF THE FIGURES

FIG. 1 shows the result of hydrolysis of corn stover pretreated at two different temperatures using the 12 different compositions of hydrolytic enzymes shown in Table 2.

### DETAILED DESCRIPTION

### Lignocellulose-Containing Material

“Lignocellulose” or “lignocellulose-containing material” means material primarily consisting of cellulose, hemicellulose, and lignin. Such material is often referred to as “biomass.”

Biomass is a complex structure of cellulose fibers wrapped in a lignin and hemicellulose sheath. The structure of biomass is such that it is not susceptible to enzymatic hydrolysis. In order to enhance enzymatic hydrolysis, the biomass has to be pre-treated in order to break the lignin seal, and solubilize the hemicellulose, and disrupt the crystalline structure of the cellulose. The cellulose can then be hydrolyzed enzymatically, e.g., by cellulolytic enzyme treatment, to convert the carbohydrate polymers into fermentable sugars which may be fermented into a desired fermentation product, such as ethanol. Hemicellulolytic enzyme treatments may also be employed to hydrolyze any remaining hemicellulose in the pre-treated biomass.

The biomass may be any material containing lignocellulose. In a preferred embodiment, the biomass contains at least about 30 wt. %, preferably at least about 50 wt. %, more preferably at least about 70 wt. %, even more preferably at least about 90 wt. % lignocellulose.

Biomass is generally found, for example, in the stems, leaves, hulls, husks, and cobs of plants or leaves, branches, and wood of trees. Biomass includes, but is not limited to, herbaceous material, agricultural residues, forestry residues, municipal solid wastes, waste paper, and pulp and paper mill residues. It is to be understood that biomass may be in the form of plant cell wall material containing lignin, cellulose, and hemicellulose in a mixed matrix. The biomass may further contain constituents, such as proteinaceous material, starch, and sugars such as fermentable or un-fermentable sugars or mixtures thereof.

Other examples of suitable biomass include corn fiber, rice straw, pine wood, wood chips, bagasse, paper and pulp processing waste, corn stover, corn cobs, hard wood such as poplar and birch, soft wood, cereal straw such as wheat straw, rice straw, switch grass, Miscanthus, rice hulls, municipal solid waste (MSW), industrial organic waste, office paper, or mixtures thereof.

In a preferred embodiment, the biomass is selected from corn stover, corn cobs, corn fiber, switch grass, wheat straw, rice straw, and bagasse, and the combination thereof.

### Pre-treatment

According to the present invention, the biomass is pre-treated chemically, mechanically, biologically, or any combination thereof prior to the hydrolysis. Also, chemical, mechanical or biological treatment may be carried out simultaneously with hydrolysis, such as simultaneously with addition of one or more cellulolytic enzymes, or other enzyme activities, to release, e.g., fermentable sugars, such as glucose or maltose.

The goal of pre-treatment is to separate or release cellulose, hemicellulose, and lignin and thus improving the rate or efficiency of hydrolysis and/or fermentation. The biomass may be present during pre-treatment in an amount between about 10-80 dry solids wt. %, preferably between about 20-70 dry solids wt. %, especially between about 30-60 dry solids wt. %, such as around about dry solids 50 wt. %.

According to the present invention, the pre-treatment is carried out at a relatively low temperature, preferably between 165° C. and 175° C., particularly from 165° C., 166° C., 167° C., 168° C., 169° C., 170° C., 171° C., 172° C., 173° C. or 174° C., and up to 166° C., 167° C., 168° C., 169° C., 170° C., 171° C., 172° C., 173° C., 174° C., or 175° C.

During a conventional biomass pre-treatment, where the temperature is usually around 180° extensive degradation of the hemicellulose components occurs. Xylan released from the lignocellulose-containing material is degraded to xylose, and xylose can be further degraded to compounds, which can be inhibitors of enzymatic hydrolysis and/or fermentation. The degradation products of xylose include but not limited to, furfural, hydroxymethyfurfural (HMF), formaldehyde, formic acid, acetaldehyde, crotonaldehyde, lactic acid, dihydroxyacetone, glyceraldehydes, pyruvaldehyde, acetol, and glycolaldehyde. Without being bound by any particular theory, it is believed that under the pre-treatment with relatively low temperature of the present invention the structure of the lignocellulose-containing material is opened but the xylan is only partially degraded. As less xylan is degraded, inhibitors are produced to a lesser extend and a washing steep can be omitted.

### Chemical Pre-treatment

The term “chemical pre-treatment” refers to any chemical pre-treatment which promotes the separation or release of cellulose, hemicellulose, or lignin. Examples of suitable chemical pre-treatment methods include treatment with, for example, dilute acid, lime, organic solvent, sulphur dioxide, or carbon dioxide. Further, wet oxidation and pH-controlled hydrothermolysis are also considered chemical pre-treatment.

In a preferred embodiment, the chemical pre-treatment is acid treatment, more preferably, a continuous dilute or mild acid treatment such as treatment with sulphuric acid, or another organic and/or inorganic acid such as acetic acid, citric acid, tartaric acid, succinic acid, hydrogen chloride or mixtures thereof. Other acids may also be used. Mild acid treatment means that the treatment pH lies in the range from about pH 1-5, preferably about pH 1-3.

In a preferred embodiment, the pre-treatment is is acid pre-treatment with 0.1 to 2.5 wt. %

acid, preferably 0.5 to 2.0 wt. % acid, preferably 0.8 to 1.5 wt. % acid. The acids for the second pre-treatment can be hydrochloric acid, phosphoric acid, sulphuric acid, sulphurous acid, carbonic acid, formic acid, acetic acid, citric acid, tartaric acid, glucuronic acid, galacturonic acid, succinic acid, and/or mixture thereof; especially sulphuric acid. The acid may be contacted with the biomass and the mixture for periods ranging from minutes to seconds. In a preferred embodiment of the present invention, the pre-treatment is is carried out for a period between 1 minutes and 60 minutes

Other chemical pre-treatment techniques are also contemplated according to the invention. It has also been shown that enzymatic hydrolysis could be greatly enhanced when the lignocellulose structure is disrupted. Ozone, organosolv (using Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al., 2005, Bioresource Technology 96: 673-686).

Other examples of suitable pre-treatment methods are described by Schell et al., 2003, Appl. Biochem and Biotechn. Vol. 105-108, p. 69-85, and Mosier et al., 2005, Bioresource Technology 96: 673-686, and U.S. Application Publication No. 2002/0164730, each of which are hereby incorporated by reference.

### Mechanical Pre-treatment

The term “mechanical pre-treatment” refers to any mechanical or physical pre-treatment which promotes the separation or release of cellulose, hemicellulose, or lignin from biomass. For example, mechanical pre-treatment includes various types of milling, irradiation, steaming/steam explosion, and hydrothermolysis.

Mechanical pre-treatment includes comminution, i.e., mechanical reduction of the size. Comminution includes dry milling, wet milling and vibratory ball milling. Mechanical pre-treatment may involve high pressure and/or elevated temperature (steam explosion). “High pressure” means pressure in the range from about 300 to 600 psi, preferably 400 to 500 psi, such as around 450 psi. Elevated temperature means temperatures in the range from about 165° C. and up to 175° C., preferably around 170° C. In a preferred embodiment, the pre-treatment of the present invention is performed as steam explosion at a temperature of 170° C. In a preferred embodiment, mechanical pre-treatment is a batch-process, with a steam gun hydrolyzer system which uses pressure and temperature as defined above. A Sunds Hydrolyzer (available from Sunds Defibrator AB (Sweden) may be used for this.

### Biological Pre-treatment

The term “biological pre-treatment” refers to any biological pre-treatment which promotes the separation or release of cellulose, hemicellulose, or lignin from the biomass. Biological pre-treatment techniques can involve applying lignin-solubilizing microorganisms. See, for example, Hsu, T.-A., 1996, Pre-treatment of biomass, in Handbook on Bioethanol: Production and Utilization, Wyman, C. E., ed., Taylor & Francis, Washington, DC, 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pre-treating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolyzates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95.

### Hydrolysis

During the hydrolysis the pre-treated biomass, preferably in the form of biomass slurry, is hydrolyzed, enzymatically and degraded into fermentable sugars or other useful compounds. As used herein, the term “biomass slurry” refers to the aqueous biomass material that undergoes enzymatic hydrolysis. Biomass slurry is produced by mixing biomass, e.g., corn stover, bagasse, etc., with water, buffer, and other pre-treatment materials. The biomass slurry may be pre-treated prior to hydrolysis or the slurry may be formed from pretreated biomass.

The dry solids content during hydrolysis may be in the range from about 5-50 wt. %, preferably about 10-40 wt. %, preferably about 20-30 wt. %. Hydrolysis may in a preferred embodiment be carried out as a fed batch process where the pre-treated biomass (i.e., the substrate) is fed gradually to, e.g., an enzyme containing hydrolysis solution.

According to the invention, hydrolysis and/or fermentation is carried out using cellulytic enzymes and a xylanase.

In a preferred embodiment, hydrolysis is carried out using a cellulolytic enzyme preparation comprising one or more polypeptides having cellulolytic enhancing activity. In a preferred embodiment, the polypeptide(s) having cellulolytic enhancing activity is of family GH61A origin. Examples of suitable and preferred cellulolytic enzyme preparations and polypeptides having cellulolytic enhancing activity are further described below.

As the biomass may contain constituents other than lignin, cellulose and hemicellulose, hydrolysis and/or fermentation may be carried out in the presence of additional enzyme activities selected from the group consisting of protease, amylase, esterase, lipase, cellulase, hemicellulase, amylase, protease, esterase, endoglucanase, beta-glucosidase, cellobiohydrolase, cellobiase, xylanase, xylose Isomerase, alpha-amylase, alpha-glucosidase, glucoamylase, and a mixture thereof

Enzymatic hydrolysis is preferably carried out in a suitable aqueous environment under conditions which can readily be determined by one skilled in the art. In a preferred embodiment, hydrolysis is carried out at suitable, preferably optimal, conditions for the enzyme(s) in question.

Preferably, hydrolysis is carried out at a temperature between 25° C. and 70° C., preferably between 40 and 60° C., especially around 50° C. Hydrolysis is preferably carried out at a pH in the range from pH 3-8, preferably pH 4-6, especially around pH 5. In addition, hydrolysis is typically carried out for between 12 and 96 hours, preferably 16 to 72 hours, more preferably between 24 and 48 hours.

Suitable process time, temperature and pH conditions can readily be determined by one skilled in the art.

### Fermentation

Fermentable sugars from pre-treated and/or hydrolyzed biomass may be fermented by one or more fermenting organisms capable of fermenting sugars, such as glucose, xylose, mannose, and galactose directly or indirectly into a desired fermentation product. The fermentation conditions depend on the desired fermentation product and fermenting organism and can easily be determined by one of ordinary skill in the art.

Especially in the case of ethanol fermentation, the fermentation may be ongoing for between 1-72 hours. In an embodiment, the fermentation is carried out at a temperature between about 20° C. to 40° C., preferably between 25° C. and 40° C., more preferably around 30° C. to around 38° C., in particular around 32° C. In one embodiment, the pH is above 5. In another embodiment, the pH is from about pH 3-7, preferably 4-6, especially between 4 and 5.

Fermentation can be carried out in a batch, fed-batch, or continuous reactor. Fed-batch fermentation may be fixed volume or variable volume fed-batch. In one embodiment, fed-batch fermentation is employed. The volume and rate of fed-batch fermentation depends on, for example, the fermenting organism, the identity and concentration of fermentable sugars, and the desired fermentation product. Such fermentation rates and volumes can readily be determined by one of ordinary skill in the art.

### Fermenting Organism

The term “fermenting organism” refers to any organism, including bacterial and fungal organisms, suitable for producing a desired fermentation product. Especially suitable fermenting organisms are able to ferment, i.e., convert, sugars, such as glucose, directly or indirectly into the desired fermentation product. Examples of fermenting organisms include fungal organisms, especially yeast. Preferred yeast includes strains of Saccharomyces spp., in particular strains of Saccharomyces cerevisiae or Saccharomyces uvarum; a strain of Pichia, preferably Pichia stipitis, such as Pichia stipitis CBS 5773; a strain of Candida, in particular a strain of Candida utilis, Candida diddensii, or Candida boidinii. Other fermenting organisms include strains of Zymomonas; Hansenula, in particular H. anomala; Klyveromyces, in particular K. fragilis; and Schizosaccharomyces, in particular S. pombe.

Commercially available yeast includes, e.g., ETHANOL RED™ yeast (available from Fermentis/Lesaffre, USA), FALI™ (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

According to the present invention, a preferred fermenting organism is able to convert C5 sugars, for example xylose, to a desired fermentation product.

### Fermentation Products

The present invention may be used for producing any fermentation product. Preferred fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B12, beta-carotene); and hormones.

Other products include consumable alcohol industry products, e.g., beer and wine; dairy industry products, e.g., fermented dairy products; leather industry products and tobacco industry products. In a preferred embodiment, the fermentation product is an alcohol, especially ethanol. The fermentation product, such as ethanol, obtained according to the invention, may preferably be used as fuel alcohol/ethanol. However, in the case of ethanol, it may also be used as potable ethanol.

### SSF, HHF and SHF

According to the invention the hydrolysis and the fermentation may be carried out simultaneously (SSF process) or sequentially (SHF process).

Hydrolysis and fermentation may be carried out as a simultaneous hydrolysis and fermentation step, or simultaneous saccharification and fermentation (SSF). In general, this means that combined/simultaneous hydrolysis and fermentation are carried out at conditions (e.g., temperature and/or pH) suitable, preferably optimal, for the fermenting organism(s) in question.

The hydrolysis step and fermentation step may be carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step).

The hydrolysis and fermentation steps may also be carried out as separate hydrolysis and fermentation, where the hydrolysis is taken to completion before initiation of fermentation. This is often referred to as “SHF”.

### Recovery

Subsequent to fermentation, the fermentation product may optionally be separated from the fermentation medium in any suitable way. For instance, the medium may be distilled to extract the fermentation product, or the fermentation product may be extracted from the fermentation medium by micro or membrane filtration techniques. Alternatively, the fermentation product may be recovered by stripping. Recovery methods are well known in the art.

### Compositions

The present invention relates to a composition of hydrolytic enzymes comprising cellulytic enzymes and a xylanase, wherein said xylanase is present in an amount of at least 10% of the total amount hydrolytic enzyme protein. The cellulytic enzymes may comprise a cellulase system derived from Trichoderma reesei.

The xylanase is preferably a GH10 xylanase. The xylanase may be present in an amount of at least 10%, at least 11%, at least 12%, at least 13%, at least 14%, at least 15%, at least 20%, or even at least 25% of the total amount of hydrolytic enzyme protein.

Preferably the hydrolytic enzymes comprise a beta-glucosidase in an amount of at least 1%, at least 2%, at least 3%, at least 4%, or even at least 5%, such as at least 6%, at least 7%, at least 8%, at least 9%, at least 10%, at least 15%, at least 20%, or even at least 25% of the total amount of hydrolytic enzyme protein.

The composition may further comprise a family 61 glycoside hydrolase, and preferably present in an amount of at least 1%, at least 2%, at least 3%, at least 4%, at least 5%, at least 6%, at least 7%, at least 8%, at least 9% at least 10%, at least 15%, at least 20%, or even at least 25% of the total amount of hydrolytic enzyme protein.

The compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the compositions of the invention. The dosage of the polypeptide composition of the invention and other conditions under which the composition is used may be determined on the basis of methods known in the art.

### Uses

The present invention also relates to methods for degrading or converting a cellulosic material, comprising: treating the cellulosic material with a composition comprising an effective amount of a polypeptide having endoglucanase activity of the present invention. In a preferred aspect, the method further comprises recovering the degraded or converted cellulosic material.

The polypeptides and host cells of the present invention may be used in the production of monosaccharides, disaccharides, and polysaccharides as chemical or fermentation feedstocks from cellulosic biomass for the production of ethanol, plastics, other products or intermediates. The composition comprising the polypeptide having endoglucanase activity may be in the form of a crude fermentation broth with or without the cells removed or in the form of a semi-purified or purified enzyme preparation. Alternatively, the composition may comprise a host cell of the present invention as a source of the polypeptide having endoglucanase activity in a fermentation process with the biomass. The host cell may also contain native or heterologous genes that encode other proteins and enzymes, mentioned above, useful in the processing of biomass. In particular, the polypeptides and host cells of the present invention may be used to increase the value of processing residues (dried distillers grain, spent grains from brewing, sugarcane bagasse, etc.) by partial or complete degradation of cellulose or hemicellulose.

### Enzymes

Even if not specifically mentioned in the context of a method or process of the invention, it is to be understood that the enzyme(s) as well as other compounds are used in an effective amount. One or more enzymes may be used.

### Cellulytic Enzymes

The term “cellulytic enzymes” or the term “cellulolytic enzyme preparation” or the term “enzymes having cellulolytic activity” as used herein is understood as comprising enzymes having cellobiohydrolase activity (EC 3.2.1.91), e.g., cellobiohydrolase I and cellobiohydrolase II, as well as endo-glucanase activity (EC 3.2.1.4) and beta-glucosidase activity (EC 3.2.1.21). The cellulolytic enzymes may, in a preferred embodiment, comprise a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

The cellulolytic enzyme preparation may comprise the commercially available product CELLUCLAST® 1.5L or CELLUZYME™ available from Novozymes A/S, Denmark or ACCELERASE™ 1000 (from Genencor Inc., USA).

The cellulolytic enzyme may be dosed in the range from 0.1-100 FPU per gram total solids (TS), preferably 0.5-50 FPU per gram TS, especially 1-20 FPU per gram TS. In another embodiment, at least 0.1 mg cellulolytic enzyme per gram total solids (TS), preferably at least 3 mg cellulolytic enzyme per gram TS, such as between 5 and 10 mg cellulolytic enzyme(s) per gram TS is(are) used for hydrolysis.

### Endoqlucanase (EG)

One or more endoglucanases may be present during hydrolysis. The term “endoglucanase” means an endo-1,4-(1,3;1,4)-beta-D-glucan 4-glucanohydrolase (E.G. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity may be determined using carboxymethyl cellulose (CMC) hydrolysis according to the procedure of Ghose, 1987, Pure and Appl. Chem. 59: 257-268.

Endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Beta-glucosidase

One or more beta-glucosidases may be present during hydrolysis. The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

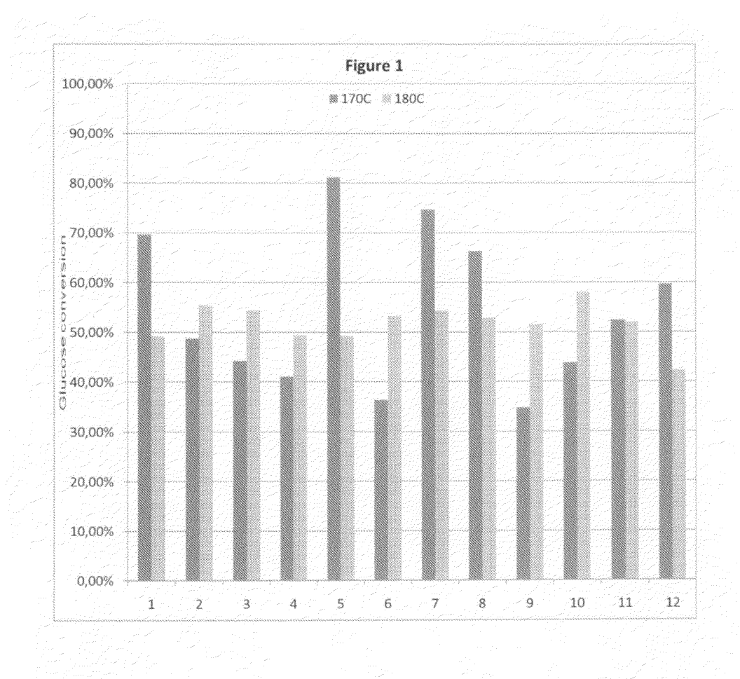

The beta-glucosidase may be of fungal origin, such as a strain of the genus Trichoderma, Aspergillus or Penicillium. The beta-glucosidase may be derived from Trichoderma reesei, such as the beta-glucosidase encoded by the bgl1 gene (see FIG. 1 of EP 562003). The beta-glucosidase may be derived from Aspergillus oryzae (recombinantly produced in Aspergillus oryzae according to WO 2002/095014), Aspergillus fumigatus (recombinantly produced in Aspergillus oryzae according to Example 22 of WO 2002/095014) or Aspergillus niger (1981, J. Appl. Vol 3, pp 157-163).

The beta-glucosidase may be derived from Penicillium brasilianum, e.g., the beta-glucosidase shown as SEQ ID NO:2 in WO 2009/111706.

### Cellobiohydrolase (CBH)

One or more cellobiohydrollases may be present during hydrolysis. The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH II from Trichoderma reseei; Humicola insolens and CBH II from Thielavia terrestris cellobiohydrolase (CELL6A).

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Family 61 Glycoside Hydrolasess

Family 61 glycoside hydrolase: The term “Family 61 glycoside hydrolase” or “Family GH61” is defined herein as a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696 as well as www.cazy.org. Presently, Henrissat lists the GH61 Family as unclassified indicating that properties such as mechanism, catalytic nucleophile/base, catalytic proton donors, and 3-D structure are not known for polypeptides belonging to this family. In the context of the present invention a family 61 glycoside hydrolase has “cellulolytic enhancing activity”.

The term “cellulolytic enhancing activity” is defined herein as a biological activity that enhances the hydrolysis of a cellulosic material by proteins having cellulolytic activity. For purposes of the present invention, cellulolytic enhancing activity is determined by measuring the increase in reducing sugars or in the increase of the total of cellobiose and glucose from the hydrolysis of a cellulosic material by cellulase protein under the following conditions: 1-50 mg of total protein/g of cellulose in PCS, wherein total protein is comprised of 80-99.5% w/w cellulase protein/g of cellulose in PCS and 0.5-20% w/w protein of cellulolytic enhancing activity for 1-7 days at 50° C. compared to a control hydrolysis with equal total protein loading without cellulolytic enhancing activity (1-50 mg of cellulolytic protein/g of cellulose in PCS). In a preferred aspect, a mixture of CELLUCLAST® 1.5L (Novozymes A/S, Bagsvaerd, Denmark) in the presence of 3% of total protein weight Aspergillus oryzae beta-glucosidase (recombinantly produced in Aspergillus oryzae according to WO 02/095014) or 3% of total protein weight Aspergillus fumigatus beta-glucosidase (recombinantly produced in Aspergillus oryzae according to Example 22 of WO 02/095014) of cellulase protein loading is used as the source of the cellulolytic activity.

### Hemicellulase

Hemicellulose can be broken down by hemicellulases and/or acid hydrolysis to release its five and six carbon sugar components. According to the present invention the pretreated lignocellulosic material is treated with one or more hemicellulases, including at least one xylanase (EC 3.2.1.8).

A xylanase for use in the present invention is an endo-1,4-beta-xylanase, and may be of Glycoside Hydrolase Family 10 or 11 (GH10 or GH11). The GH10 or GH11 are defined in Cantarel et al. (2008) in Nucl. Acids Res. 2009 37: D233-D238 and on www.cazy.org.

The xylanase may be of any origin including mammalian, plant or animal origin;

however, it is preferred that the xylanase is of microbial origin. In particular the xylanase may be one derivable from a filamentous fungus or a yeast. Preferably the xylanase is derived from a filamentous fungus such as from Aspergillus sp., Bacillus sp., Humicola sp., Myceliophotora sp., Poitrasia sp. Rhizomucor sp. or Trichoderma.

Preferred is a xylanase a GH10 xylanase. A suitable GH10 xylanase may be derived from Aspergillus aculeatus such as the enzyme disclosed as xylanase II in SEQ ID NO:2 of WO 1994/021785. Also preferred are xylanases having at least 50% identity, at least 60% identity, at least 70% identity, at least 80% identity, at least 90% identity, at least 95% identity, or even at least 99% identity with the amino acid sequence shown in SEQ ID NO:2 of WO 1994/021785 and/or as SEQ ID NO:1 herein.

Examples of commercial xylanases include SHEARZYME™ and BIOFEED WHEAT™ from Novozymes NS, Denmark.

According to the present invention the xylanase is added to and/or present in the hydrolysis step of the present invention in an amount of at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or even at least 25% of the total amount of hydrolytic enzyme protein.

The xylanase is added to and/or present in the hydrolysis step of the present invention in an amount of 0.001-1.0 g/kg DS substrate, preferably in the amounts of 0.005-0.5 g/kg DS substrate, and most preferably in an amount of 0.05-0.10 g/kg DS substrate.

The hemicellulase must be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

Other preferred hemicellulases include arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes A/S, Denmark).

### Xylose Isomerase

Xylose isomerases (D-xylose ketoisomerase) (E.C. 5.3.1.5.) are enzymes that catalyze the reversible isomerization reaction of D-xylose to D-xylulose.

A xylose isomerase may be used in the method or process and may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus and Flavobacterium, and Thermotoga, including T. neapolitana (Vieille et al., 1995, Appl. Environ. Microbiol. 61 (5), 1867-1875) and T. maritime. Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988, Agric. Biol. Chem., 52 (7): 1817-1824. The xylose isomerase may preferably be derived from a strain of Candida boidinii (Kloeckera 2201), deposited as DSM 70034 and ATCC 48180, disclosed in Ogata et al., Agric. Biol. Chem, Vol. 33, p. 1519-1520 or Vongsuvanlert et al., 1988, Agric. Biol. Chem, 52 (2), p. 1519-1520.

In one embodiment, the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Patent No. 4,351,903, U.S. Patent No. 4,137,126, U.S. Pat. No. 3,625,828, HU patent no. 12,415, DE patent 2,417,642, JP patent no. 69,28,473, and WO 2004/044129, each incorporated by reference herein. The xylose isomerase may be either in immobilized or liquid form. Liquid form is preferred. Examples of commercially available xylose isomerases include SWEETZYME™ T from Novozymes A/S, Denmark. The xylose isomerase is added in an amount to provide an activity level in the range from 0.01-100 IGIU per gram total solids.

### Alpha-Amylase

One or more alpha-amylases may be used. Preferred alpha-amylases are of microbial, such as bacterial or fungal origin. The most suitable alpha-amylase is determined based on process conditions but can easily be done by one skilled in the art.

The preferred alpha-amylase may be an acid alpha-amylase, e.g., fungal acid alpha-amylase or bacterial acid alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

An acid alpha-amylases may according to the invention be added in an amount of 0.1 to 10 AFAU/g DS, preferably 0.10 to 5 AFAU/g DS, especially 0.3 to 2 AFAU/g DS.

Preferred commercial compositions comprising alpha-amylase include MYCOLASE from DSM, BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes NS, Denmark).

### Proteases

A protease may be added during hydrolysis, fermentation or simultaneous hydrolysis and fermentation. The protease may be added to deflocculate the fermenting organism, especially yeast, during fermentation. The protease may be any protease. In a preferred embodiment, the protease is an acid protease of microbial origin, preferably of fungal or bacterial origin. An acid fungal protease is preferred, but also other proteases can be used.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Mucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze et al., 1964, Agr. Biol. Chem. Japan, 28, 216), Aspergillus saitoi (see, e.g., Yoshida, 1954, J. Agr. Chem. Soc. Japan, 28, 66), Aspergillus awamori (Hayashida et al., 1977, Agric. Biol. Chem., 42 (5), 927-933, Aspergillus aculeatus (WO 1995/02044), or Aspergillus oryzae, such as the pepA protease; and acidic proteases from Mucor pusillus or Mucor miehei.

Also contemplated are neutral or alkaline proteases, such as a protease derived from a strain of Bacillus. For example, protease contemplated for the invention is derived from Bacillus amyloliquefaciens and has the sequence obtainable at Swissprot as Accession No. P06832. Also contemplated are the proteases having at least 90% identity to amino acid sequence obtainable at Swissprot as Accession No. P06832 such as at least 92%, at least 95%, at least 96%, at least 97%, at least 98%, or particularly at least 99% identity.

Further contemplated are the having at least 90% identity to the Thermoascus aurantiacus protease disclosed as SEQ ID NO:1 in WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment, the protease may be a protease preparation derived from a strain of Aspergillus, such as Aspergillus oryzae. In another embodiment, the protease may be derived from a strain of Rhizomucor, preferably Rhizomucor meihei. In another contemplated embodiment, the protease may be a protease preparation, preferably a mixture of a proteolytic preparation derived from a strain of Aspergillus, such as Aspergillus oryzae, and a protease derived from a strain of Rhizomucor, preferably Rhizomucor meihei.

Aspartic acid proteases are described in, for example, Hand-book of Proteolytic Enzymes, Edited by A. J. Barrett, N. D. Rawlings and J. F. Woessner, Aca-demic Press, San Diego, 1998, Chapter 270). Suitable examples of aspartic acid protease include, e.g., those disclosed in R. M. Berka et al., Gene, 96, 313 (1990)); (R. M. Berka et al., Gene, 125, 195-198 (1993)); and Gomi et al., Biosci. Biotech. Biochem. 57, 1095-1100 (1993), which are hereby incorporated by reference. Commercially available products include ALCALASE®, ESPERASE™, FLAVOURZYME™, PROMIX™, NEUTRASE®, RENNILASE®, NOVOZYM™ FM 2.0L, and NOVOZYM™ 50006 (available from Novozymes A/S, Denmark) and GC106™ and SPEZYME™ FAN from Genencor Int., Inc., USA.

The protease may be present in an amount of 0.0001-1 mg enzyme protein per g DS, preferably 0.001 to 0.1 mg enzyme protein per g DS. Alternatively, the protease may be present in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

### Lipolytic Enzyme

A lipolytic enzyme is an enzyme which is capable of hydrolyzing carboxylic ester bonds to release a carboxylic acid or carboxylate (EC 3.1.1). Said lipolytic enzymes primarily comprise the following three subtypes: galactolipases (EC 3.1.1.26), phospholipases (Al or A2, EC 3.1.1.32 or 3.1.1.4) and triacylglycerol lipases (EC 3.1.1.3), predominantly having activity for galactolipids, phospholipids, and triglyceride, respectively. The activities may be determined by any suitable method, e.g. by assays known in the art or described later in this specification.

The lipolytic enzyme may have a narrow specificity with activity for one of the three substrates and little or no activity for the other two, or it may have a broader specificity with predominant activity for one substrate and less activity for the other two substrates. A combination of two or more lipolytic enzymes may be used.

The lipolytic enzymes may be prokaryotic, particularly bacterial, or eukaryotic, e.g. from fungal or animal sources. Lipolytic enzymes may be derived, e.g. from the following genera or species: Thermomyces, T. lanuginosus (also known as Humicola lanuginosa); Humicola, H. insolens; Fusarium, F. oxysporum, F. solani, F. heterosporum; Aspergillus, A. tubigensis, A. niger, A. oryzae; Rhizomucor; Candida, C. antarctica, C. rugosa, Penicillium, P. camembertii; Rhizopus, Rhizopus oryzae; Absidia. Dictyostelium, Mucor, Neurospora, Rhizopus, R. arrhizus, R. japonicus, Sclerotinia, Trichophyton, Whetzelinia, Bacillus, Citrobacter, Enterobacter, Edwardsiella, Erwinia, Escherichia, E. coli, Klebsiella, Proteus, Providencia, Salmonella, Serratia, Shigella, Streptomyces, Yersinia, Pseudomonas, P. cepacia.

The invention described and claimed herein is not to be limited in scope by the specific embodiments herein disclosed, since these embodiments are intended as illustrations of several aspects of the invention. Any equivalent embodiments are intended to be within the scope of this invention as well as combinations of one or more of the embodiments. Various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties. The present invention is further described by the following examples which should not be construed as limiting the scope of the invention.

### Materials and Methods

### Determination of Identity

The relatedness between two amino acid sequences or between two nucleotide sequences is described by the parameter “identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

### Enzymes

Cellulase preparation C comprising a cellulolytic enzyme complex derived from Trichoderma reesei, a T. aurantiacus polypeptide having cellulolytic enhancing activity (GH61A) shown in WO2005074656 as amino acids 23 to 250 of SEQ ID NO: 2, and an Aspergillus fumigatus beta-glucosidase shown in WO05047499 as amino acids 1-863 of SEQ ID NO: 2.

A preparation comprising the Aspergillus aculeatus xylanase disclosed as Xyl II in SEQ ID NO:2 of W09421785 and as SEQ ID NO:1 herein.
A preparation comprising a T. aurantiacus polypeptide having cellulolytic enhancing activity (GH61A) shown in WO2005074656 as amino acids 23 to 250 of SEQ ID NO: 2.
A preparation comprising Penicillium brasilianum beta-glucosidase shown as SEQ ID NO:2 in WO 2009/111706.

### EXAMPLES

### Example 1

Pre-treatment: Corn stover was soaked in 1.13 wt. % H₂SO₄ solution at ambient temperature for 4 hours. The corn stover was subjected to steam explosion for 5.5 minutes at 170° C. and 180° C., respectively. The composition of pretreated the corn stover (PCS) was analyzed and the results are shown in Table 1.

Enzyme compositions 1-12 (table 2) were test on the two batches of PCS. The hydrolysis of the PCS was evaluated in a system with 12.6% initial TS, 20 g total weight. The enzyme dosage was 5 mg enzyme protein/g cellulose. Hydrolysis time was 72 hrs with 130 rpm at 50° C. and pH 5.0. Glucose content in hydrolysate was analyzed by HPLC. Cellulose conversion was calculated as the percentage of produced glucose out of the total glucose potential in PCS. All treatments were preformed in 3 replications The results are shown in FIG. 1.

Treatments with high xylanase dosage show improved hydrolysis on biomass pretreated at low temperature. No effect of high xylanase dosage is seen on the biomass pretreated at high temperature

TABLE 1
The composition of PCS analyzed according to the NREL
Laboratory Analytical Procedure Version 2006,
sections 10.4 and 10.11.

| Pretreatment temperature | Glucan | Xylan | Total solids |
|---|---|---|---|
| 170° C. | 36.72% | 1.79 | 18.41% |
| 180° C. | 36.16 | 1.30 | 19.71% |

TABLE 2
Enzyme compositions NO. 1 to 12. Cell. Prep.

| NO. | Cell. Prep. C | Xyl II | GH61 | BG |
|---|---|---|---|---|
| 1 | 0.65 | 0.15 | 0.05 | 0.15 |
| 2 | 0.65 | 0.05 | 0.15 | 0.15 |
| 3 | 0.75 | 0.05 | 0.15 | 0.05 |
| 4 | 0.65 | 0.05 | 0.05 | 0.25 |
| 5 | 0.65 | 0.25 | 0.05 | 0.05 |
| 6 | 0.85 | 0.05 | 0.05 | 0.05 |
| 7 | 0.65 | 0.15 | 0.15 | 0.05 |
| 8 | 0.75 | 0.15 | 0.05 | 0.05 |
| 9 | 0.75 | 0.05 | 0.05 | 0.15 |
| 10 | 0.65 | 0.05 | 0.25 | 0.05 |
| 11 | 1 | | | |
| 12 | 0.85 | 0.15 | | |

C = Cellulase preparation C,
Xyl II = A. aculeatus xylanase,
GH 61 = T. aurantiacus GH61;
BG = Penicillium brasilianum beta-glucosidase.

1-15. (canceled)

16. A process for producing a hydrolysate of a lignocellulosic material comprising
a) subjecting the lignocellulosic material to a pretreatment at a temperature between 165° C. and 175° C., and
b) subjecting the pretreated lignocellulosic material to the action of hydrolytic enzymes to produce a hydrolysate,
wherein the hydrolytic enzymes comprise cellulytic enzymes and a xylanase, and wherein said xylanase is present in an amount of at least 10% of the total amount hydrolytic enzyme protein.

17. The process according to claim 16, wherein the hydrolytic enzymes comprise a cellulase system derived from Trichoderma reesei.

18. The process according to claim 16, wherein the xylanase has at least 90% identity with the sequence shown as SEQ ID NO:1.

19. The process according to claim 16, wherein the xylanase is present in an amount of at least 15% of the total amount of hydrolytic enzyme protein.

20. The process according to claim 16, wherein the hydrolytic enzymes comprise a family 61 glycoside hydrolase in an amount of at least 5% of the total amount of hydrolytic enzyme protein.

21. The process according to claim 16, wherein the hydrolytic enzymes comprise a beta-glucosidase in an amount of at least 1% of the total amount of hydrolytic enzyme protein.

22. The process according to claim 16, wherein the lignocellulose-containing material originates from materials selected from the group consisting of corn stover, corn fiber, hard wood, such as poplar and birch, soft wood, cereal straw, such as, wheat straw, switch grass, rice hulls, municipal solid waste, industrial organic waste, office paper, and mixtures thereof.

23. The process according to claim 16, wherein the pretreatment in step (a) is carried out using an organic or inorganic acid selected from the group consisting of sulphuric acid, acetic acid, citric acid, tartaric acid, succinic acid, and mixtures thereof.

24. The process according to claim 16, wherein the pretreatment in step (a) is carried out using from 0.1 to 2.5 wt. % acid.

25. The process according to claim 16, wherein the pretreatment is performed at a temperature of around 170° C.

26. The process according to claim 16, wherein the pretreatment is performed as steam explosion.

27. The process according to claim 16, further comprising fermenting the hydrolysate of step (b) to produce a fermentation product.

28. The process according to claim 27, wherein the hydrolysis and fermentation are simultaneous.

29. The process according to claim 27, further comprising recovery of the fermentation product.
